SEQUENCE LISTING (12 sequences)

SEQ ID NO: 1 - protein, 10 aa, synthetic construct
EGTRPGPPGP

SEQ ID NO: 2 - protein, 10 aa, synthetic construct
TYPGRHGPPG

SEQ ID NO: 3 - protein, 11 aa, synthetic construct
SEGTRPGPPGP

SEQ ID NO: 4 - protein, 9 aa, synthetic construct
GTRPGPPGP

SEQ ID NO: 5 - protein, 11 aa, synthetic construct
VTYPGRHGPPG

SEQ ID NO: 6 - protein, 9 aa, synthetic construct
YPGRHGPPG

SEQ ID NO: 7 - protein, 10 aa, synthetic construct
  SITE 10: note = linked to biotin via optional linker
EGTRPGPPGP

SEQ ID NO: 8 - protein, 10 aa, synthetic construct
  SITE 10: note = linked to biotin via optional linker
TYPGRHGPPG

SEQ ID NO: 9 - protein, 13 aa, synthetic construct
  SITE 13: note = linked to keyhole limpet hemocyanin
EGTRPGPPGPGGC

SEQ ID NO: 10 - protein, 13 aa, synthetic construct
  SITE 13: note = linked to keyhole limpet hemocyanin
TYPGRHGPPGGGC

SEQ ID NO: 11 - protein, 11 aa, synthetic construct
  SITE 11: note = biotinylated
EGTRPGPPGPK

SEQ ID NO: 12 - protein, 11 aa, synthetic construct
  SITE 11: note = biotunylated
TYPGRHGPPGK

(19) United States
(12) Patent Application Publication
(10) Pub. No.: US 2026/0219282 A1
(43) Pub. Date: Jul. 30, 2026

(54) C4A3-HNE and C4A4-HNE Assay

(71) Applicant: Nordic Bioscience A/S, Herlev (DK)

(72) Inventors: Joachim Høg Mortensen, Hundige Strand (DK); Marta Alexdottir, Herlev (DK); Morten Asser Karsdal, København Ø (DK); Anne-Christine Bay-Jensen, København S (DK)

(73) Assignee: Nordic Bioscience A/S, Herlev (DK)

(21) Appl. No.: 19/152,230

(22) PCT Filed: Jan. 30, 2024
(86) PCT No.: PCT/EP2024/052255

§ 371 Date: Jul. 30, 2025

(30) Foreign Application Priority Data
Jan. 31, 2023 (GB) 2301401.2

(51) Int. Cl. G01N 33/68; C07K 16/18; G01N 33/543

(52) U.S. Cl. CPC G01N 33/6893; C07K 16/18; G01N 33/54386; C07K 2317/32; C07K 2317/33; G01N 2470/04; G01N 2470/10; G01N 2800/065

(57) ABSTRACT

Disclosed herein are methods of immunoassay for detecting HNE-generated fragments of the α3 chain or α4 chain of type IV collagen in a patient sample, and the use thereof for detecting and/or monitoring inflammatory bowel disease (IBD) or a particular level of severity thereof in a patient. Also disclosed are monoclonal antibodies and assay kits for use in the methods of immunoassay.

### FIELD OF THE INVENTION

The present invention relates to methods of immunoassay for detecting HNE-generated fragments of the α3 chain or α4 chain of type IV collagen in a patient sample, and the use thereof for detecting and/or monitoring inflammatory bowel disease (IBD) or a particular level of severity thereof in a patient. The present invention also relates to monoclonal antibodies and assay kits for use in said methods of immunoassay.

### BACKGROUND

Inflammatory bowel disease (IBD) is a chronic immune-mediated disease of the gastrointestinal (GI) tract, encompassing Crohn's disease (CD) and ulcerative colitis (UC). These two diseases differ by their location and depth of involvement; UC involves superficial inflammation of the colonic mucosa, while CD causes transmural ulceration of any portion of the GI tract. IBD develops in genetically susceptible individuals due to an abnormal immune response to the gut microbiota, where excessive inflammatory reactions lead to a continued deterioration of the intestinal barrier ^{[1]}. Immune cells are considered to be the main pathogenic factor in IBD, as they initiate and sustain inflammation by expressing and secreting a plethora of different cytokines and proteases ^{[2]}. For this reason, immune cells are the key targets of biologic treatments in IBD. Although disease management has substantially improved with the introduction of anti-tumor necrosis factor (TNF) antibodies, up to 40% of patients with IBD experience non-response or loss of response after induction therapy ^{[3]}. Better patient profiling and monitoring is therefore needed in order to provide optimal treatment.

Improving the detection of subclinical ulceration is essential since early-stage IBD presents a unique window of opportunity for intervention, as the disease becomes a self-sustaining process once deep inflammation becomes established.

Neutrophils are the most abundant immune cells in circulation, the first responders of the innate immune system, and are best known for their rapid recruitment to sites of inflammation. Upon activation, neutrophils produce NETs (neutrophil extracellular traps) where they release the antimicrobial protein Calprotectin, and proteases such as human neutrophil elastase (HNE) from granules stored in their cytoplasm ^{[4]}. Constant activation and excessive recruitment of neutrophils is a common feature in IBD and their infiltration into the intestinal tissue has been correlated to disease activity ^{[4]}. HNE is a serine protease and is one of the most abundant proteins in NETs. HNE levels are elevated in the plasma of IBD patients compared to healthy subjects, HNE expression is upregulated in the UC intestinal tissue, and NE levels have been correlated with histopathological disease scores in CD ^{[5-7]}.

As previously mentioned, disruption of the intestinal barrier is a hallmark of IBD as it leads to the exposure of intestinal microbes, furthering the initial inflammation. Therefore, the extracellular matrix (ECM) is of high importance for structural integrity of the intestinal tissue—especially the intestinal basement membrane (BM). Acting as a front-line defense, the intestinal BM is a collagen rich matrix with type IV collagen being the major collagenous component ^{[8]}. Type IV collagen consists of six genetically distinct alpha chains, with three chains forming a singular collagen molecule. Three different α-chain compositions are known for type IV collagen, and they differ between tissues. The composition α1α1α2 is thought to be present in almost all basement membranes, and α5α5α6 is mainly localized in the epidermis. The third composition, α3α4α5, was only thought to be present in the kidney and alveoli of the lung (both organs require selective filtration through their BM) ^{[9]}. However, immunostainings of all six alpha chains in the human colon show that these chains are also present in the intestines, with α3 and α4 being solely expressed on the surface tip of the mucosa facing the lumen of the GI tract ^{[10,11]}.

### SUMMARY OF THE INVENTION

The applicant has now developed and biologically evaluated two competitive enzyme-linked immunosorbent assays (ELISAs) targeting HNE-generated neo-epitopes of the α3 or α4 chain of type IV collagen, and has demonstrated the utility of these HNE-generated neo-epitopes as biomarkers for inflammatory bowel disease (IBD) in general, and early-stage or mild IBD in particular.

Accordingly, in a first aspect the present invention provides a method of immunoassay, said method comprising contacting a patient sample with a monoclonal antibody that specifically binds to an HNE-generated neo-epitope of an HNE-generated fragment of the α3 or α4 chain of type IV collagen, and detecting and determining the amount of binding between the monoclonal antibody and peptides in the sample, wherein the HNE-generated neo-epitope consists of an N-terminus or C-terminus sequence of the HNE-generated fragment at an end of the HNE-generated fragment that has been cleaved by HNE.

In a preferred embodiment, the method of immunoassay comprises;
i) contacting a patient sample with a monoclonal antibody that specifically binds to the N-terminus amino acid sequence EGIRPGPPGP (SEQ ID NO: 1) (also referred to herein as “C4A3-HNE”, or the “C4A3-HNE target sequence”) or that specifically binds to the N-terminus amino acid sequence TYPGRHGPPG (SEQ ID NO: 2) (also referred to herein as “C4A4-HNE”, or the “C4A4-HNE target sequence”); and
ii) detecting and determining the amount of binding between said monoclonal antibody and peptides in the sample.

In a preferred embodiment, the method of immunoassay is a method of immunoassay for detecting and/or monitoring inflammatory bowel disease or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the inflammatory bowel disease (IBD) is Crohn's disease (CD) or ulcerative colitis (UC).

In a preferred embodiment, the method is a method of immunoassay for detecting mild or early stage inflammatory bowel disease.

In a preferred embodiment, the patient sample is a human biofluid sample. Preferably the sample is a blood-based sample, such as blood (whole blood), plasma or serum.

Where the monoclonal antibody is a monoclonal antibody that specifically binds to the N-terminus amino acid sequence EGTRPGPPGP (SEQ ID NO: 1), the monoclonal antibody preferably does not specifically bind to a peptide having the N-terminus amino acid sequence SEGTRPGPPGP (SEQ ID NO: 3) (i.e. an elongated version of the C4A3-HNE target sequence extended at its N-terminus by the addition of a serine residue) and/or does not specifically bind to a peptide having the N-terminus amino acid sequence GTRPGPPGP (SEQ ID NO: 4) (i.e. a truncated version of the C4A3-HNE target sequence truncated by removal of the first glutamic acid residue). Preferably, the ratio of the affinity of said antibody for the C4A3-HNE target sequence to the affinity of said antibody for the elongated version of the target sequence is at least 10 to 1, and more preferably is at least 20 to 1 or at least 30 to 1.

Where the monoclonal antibody is a monoclonal antibody that specifically binds to the N-terminus amino acid sequence TYPGRHGPPG (SEQ ID NO: 2), the monoclonal antibody preferably does not specifically bind to a peptide having the N-terminus amino acid sequence VTYPGRHGPPG (SEQ ID NO: 5) (i.e. an elongated version of the C4A4-HNE target sequence extended at its N-terminus by the addition of a valine residue) and/or does not specifically bind to a peptide having the N-terminus amino acid sequence YPGRHGPPG (SEQ ID NO: 6) (i.e. a truncated version of the C4A4-HNE target sequence truncated by removal of the first threonine acid residue). Preferably, the ratio of the affinity of said antibody for the C4A4-HNE target sequence to the affinity of said antibody for the elongated version of the target sequence is at least 10 to 1, and more preferably is at least 20 to 1 or at least 30 to 1.

Where the monoclonal antibody is a monoclonal antibody that specifically binds to the N-terminus amino acid sequence EGTRPGPPGP (SEQ ID NO: 1), the monoclonal antibody may for example be raised against a synthetic peptide having the N-terminus amino acid sequence EGTRPGPPGP (SEQ ID NO: 1); and where the monoclonal antibody is a monoclonal antibody specifically binds to the N-terminus amino acid sequence TYPGRHGPPG (SEQ ID NO: 2), the monoclonal antibody may for example be raised against a synthetic peptide having the N-terminus amino acid sequence TYPGRHGPPG (SEQ ID NO: 2). For example, the monoclonal antibodies may be raised by: (a) immunizing a rodent (or other suitable mammal) with a synthetic peptide comprising the N-terminus sequence EGTRPGPPGP (SEQ ID NO: 1) or TYPGRHGPPG (SEQ ID NO: 2), which peptide may optionally be linked at its C-terminus to an immunogenic carrier protein (such as keyhole limpet hemocyanin); (b) isolating and cloning a single antibody producing cell; and (c) assaying the resulting monoclonal antibodies to ensure that they have the desired specificity.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′) 2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method can be used for measuring the amount of binding. For example, an ELISA method can be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. IBD) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without IBD); and the term “values associated with known disease severity” means standardised quantities of binding determined by the method described supra for samples from patients known to have disease (i.e. an IBD) of a known severity.

In a second aspect, the present invention provides a method of treating inflammatory bowel disease (IBD) in a patient in need thereof, the method comprising:
(a) carrying out a method of immunoassay for detecting IBD or a particular level of severity thereof in accordance with the first aspect of the present invention on a sample from a patient; and
(b) administering to the patient a therapy for the treatment of IBD if it is determined in step (a) that the patient has IBD or said particular level of severity thereof.

Preferred embodiments of the method in accordance with the second aspect will be apparent from the foregoing discussion of preferred embodiments of the methods according to the first aspect. For example, the IBD may in particular comprise Crohn's disease (CD) or ulcerative colitis (UC); and/or step (a) may in particular comprise carrying out the method of immunoassay in accordance with the first aspect of the invention for detecting mild or early stage IBD and step (b) may then comprise administering to the patient a therapy for the treatment of IBD if it is determined in step (a) that the patient has mild or early stage IBD.

The therapy may be any therapy suitable for treating the inflammatory bowel disease in question. The therapy may for example comprise or consist of one or more medicaments, one or more lifestyle changes, one or more surgeries or combinations thereof. Medicaments may be formulated for topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, palliative surgeries and/or restorative surgeries.

For example, where the inflammatory bowel disease is Crohn's disease suitable therapies may comprise one or more of: lifestyles changes such as dietary adjustments, elemental diet, proper hydration, smoking cessation; medicaments such as antibiotics, aminosalicylate anti-inflammatory corticosteroids, 5-aminosalicylic acid (5-ASA), prednisone, azathioprine, 6-mercaptopurine, methotrexate, anti-TNF therapies (also referred to in the art as INF inhibitors) such as monoclonal antibodies that inhibit the effects of INF such as infliximab, adalimumab and certolizumab, and other biologics and antibodies such as vedolizumab, ustekinumab, and natalizumab; and combinations thereof.

Where the inflammatory bowel disease is ulcerative colitis suitable therapies may comprise one or more of: medicaments such as aminosalicylates such as Mesalazine, Sulfasalazine, Balsalazide, Olsalazine, corticosteroids such as Cortisone, Prednisone, Hydrocortisone, Methylprednisolone, Budesonide, immunosuppressive drugs such as Mercaptopurine, Azathioprine, Methotrexate; and anti-TNF therapies (INF inhibitors) such as Infliximab, Adalimumab and Golimumab, and other biologics and antibodies such as Tofacitinib and Vedolizumab; surgical procedures such as a partial or total colectomy; and combinations thereof.

In a third aspect, the present invention provides a monoclonal antibody that specifically binds to the N-terminus amino acid sequence EGTRPGPPGP (SEQ ID NO: 1) (i.e. the C4A3-HNE target sequence) or that specifically binds to the N-terminus amino acid sequence TYPGRHGPPG (SEQ ID NO: 2) (i.e. the C4A4-HNE target sequence).

The antibody according to the third aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

In a fourth aspect, the present invention provides an immunoassay kit comprising a monoclonal antibody in accordance with the third aspect of the present invention, and at least one of:
a streptavidin coated well plate
a biotinylated peptide EGTRPGPPGP-L-Biotin (SEQ ID NO: 7) wherein L is an optional linker, or a biotinylated peptide TYPGRHGPPG-L-Biotin (SEQ ID NO: 8) wherein L is an optional linker
a secondary antibody for use in a sandwich immunoassay
a calibrator protein comprising the N-terminus amino acid sequence EGTRPGPPGP (SEQ ID NO: 1) or a calibrator protein comprising the N-terminus amino acid sequence TYPGRHGPPG (SEQ ID NO: 2)
an antibody biotinylation kit
an antibody HRP labelling kit
an antibody radiolabelling kit

The immunoassay kit according to the fourth aspect of the invention is, in particular, suitable for use in carrying out the method of immunoassay according to the first aspect of the invention. Further preferred embodiments and features of the immunoassay kit according to the fourth aspect will therefore be apparent from the above discussion of the preferred embodiments of the methods according to the first aspect.

### FIGURES

FIG. 1: Analysis of dose-dependent reactivity for the C4A3-HNE and C4A4-HNE monoclonal antibodies against the selection peptides, elongated peptides, and truncated peptides.

FIG. 2: Results of HNE digestion of synthetic peptides mimicking the (A) type IV collagen alpha-3 chain and (B) type IV collagen alpha-4 chain. Peptides were incubated with active HNE for 4 hours. Negative controls were peptide (without protease) and protease (without peptide). HNE digestion of both peptides released the neo-epitopes upon incubation. Buffer was used as a background control, shown values are background subtracted.

FIG. 3: Measurement of C4A3-HNE in CD and UC. C4A3-HNE is elevated in both CD and UC compared to healthy controls (p<0.05). Data is presented as median.

FIG. 4: Measurement of C4A3-HNE in different stages of IBD. High levels of C4A3-HNE reflect early mucosal damage in patients with IBD.

FIG. 5: Measurement of FeCal and CRP in UC and CD. Inflammatory markers are lower in IBD patients with high levels of C4A3-HNE.

FIG. 6: Measurement of C4A3-HNE and C4Ma3 in lung disease. The two immune-cell generated COL4 alpha-3 chain biomarkers do not reflect the same pathological process reflected by a weak correlation of 0.33.

FIG. 7: Measurement of C4A4-HNE in CD and UC. C4A4-HNE is elevated in IBD compared to healthy.

FIG. 8: Measurement of C4A4-HNE in CD and UC. C4A4-HNE is significantly elevated in both CD and UC compared with healthy donors (****=p<0.0001, **=p<0.01).

FIG. 9: Measurement of C4A4-HNE in different stages of IBD. C4A4-HNE is numerically higher in patients with mild endoscopic UC, dose-dependently decreasing across patients with moderate and severe endoscopic UC.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Assay Development

Neo-Epitope Identification Through in-Silico Cleavage

Based on the known expression patterns of the α3 and α4 chains of type IV collagen in the intestines, with these chains being solely expressed on the surface tip of the mucosa facing the lumen of the GI tract ^{[10,11]}, the applicant hypothesized that these chains are readily accessible to all initial inflammatory insults such as immune cell secreted ECM-degrading proteases. With neutrophils being recruited rapidly to novel sites of inflammation, the presence of HNE can also be linked to initial insult response. The applicant therefore hypothesized that HNE-generated neo-epitopes of the α3 and α4 chains of type IV collagen might function as biomarkers reflecting early tissue injury in IBD, aiding in the implementation of prompt treatment.

Initial neo-epitope identification was done through in-silico cleavage of the alpha-3 and alpha-4 chains of type IV collagen, based on the cleavage sites of HNE. Cleavage sites are at the C-terminal of Ala, Val and Ile, and based on in-house mass spec data HNE also seems to cleave at the C-terminal of Serine. Cleavage fragments were first generated manually, and then verified using a pre-made in-silico cleavage algorithm through the software Rapid Peptide Generator ^{[12]}(RPG, Python v3.9). Fragments <10 amino acids were avoided, as well as proline-glycine repeats. In-house MS databases were scanned to prevent the fragments of interest being produced naturally by other proteases or as a passive byproduct. One neo-epitope fragment was chosen for the HNE-degraded alpha-3 chain, comprising the N-terminus sequence EGTRPGPPGP (SEQ ID NO: 1) (“C4A3-HNE”) formed after cleavage of the alpha-3 chain after residue 1059 of the intact chain, and another neo-epitope fragment was chosen for the HNE-degraded alpha-4 chain, comprising the N-terminus sequence TYPGRHGPPG (SEQ ID NO: 2) (“C4A4-HNE”) formed after cleavage of the alpha-4 chain after residue 670 of the intact chain.

Monoclonal Antibody Production, Characterization of Clones and their Specificity

Female Balb/C mice of 6-7 weeks of age were immunized subcutaneously with 200 μl emulsified antigen and 100 μg of the C4A3-HNE (EGTRPGPPGP-GGC-KLH (SEQ ID NO: 9)) or C4A4-HNE (TYPGRHGPPG-GGC-KLH (SEQ ID NO: 10)) immunogenic peptide (KLH=keyhole limpet hemocyanin). Immunizations were performed consecutively at 2-week intervals until stable sera titer levels were reached. The mouse with the highest titer was selected for fusion and rested for one month. The mouse was subsequently boosted intravenously with 50 μg of the respective immunogenic peptide in 100 μl of 0.9% NaCl solution, three days before isolation of the spleen for cell fusion. Hybridoma cells were produced by fusion of the mouse spleen cells with SP2/0 myeloma cells as described by Grefter et al ^{[13]}. The hybridoma cells were cloned using a semi-solid medium method and transferred into a 96-well microtiter plates for further growth where the limiting dilution method was applied to promote monoclonal growth. Supernatants were screened for reactivity to the respective neo-epitopes using an indirect ELISA with streptavidin-coated microtiter plates and biotinylated peptides for C4A3-HNE (EGTRPGPPGP-K-Biotin (SEQ ID NO: 11)) or C4A4-HNE (TYPGRHGPPG-K-Biotin (SEQ ID NO: 12)). Specificities of the clones to the standard/selection peptides (EGTRPGPPGP (SEQ ID NO: 1) for C4A3-HNE or TYPGRHGPPG (SEQ ID NO: 2) for C4A4-HNE), elongated peptides (SEGTRPGPPGP (SEQ ID NO: 3) for C4A3-HNE or VTYPGRHGPPG (SEQ ID NO: 5) for C4A4-HNE) and truncated peptides (GTRPGPPGP (SEQ ID NO: 4) for C4A3-HNE or YPGRHGPPG (SEQ ID NO: 6) for C4A4-HNE) were tested. Cross-reactivity between C4A3-HNE and C4A4-HNE was also tested. The supernatants were purified using HiTrap affinity columns (GE Healthcare Life Science, Little Chalfront, Buckinghamshire, UK) according to manufacturer's instructions. Antibodies were then further labeled with activated peroxidase from horse radish with a Peroxidase Labeling Kit, according to the kit protocol (Roche Diagnostics, Mannheim, Germany, CAT no. 11829696001).

### ELISA Protocol

Preliminary experiments were carried out where the assay reagents, their concentrations and incubation periods were optimized. The final competitive ELISA procedures are as follows: A 96-well streptavidin plate was coated with synthetic biotinylated peptide (100 μl/well) diluted in assay buffer (25 mM PBS-BTB 6 g NaCl/L, pH 7.4 for C4A3-HNE and 25 mM PBS-BTB 8 g NaCl/L, pH 7.4 for C4A4-HNE), and incubated for 30 min at 20° C. in the dark while shaking at 300 rpm. After each incubation step, the plates were washed with washing buffer (25 mM Tris, 50 mM NaCl, 0.1% (v/v) Tween-20, pH 7.2) using a standardized ELISA plate washer (BioTek Instruments, Microplate washer, Elx405 Select CW). Standards and serum samples (20 μl/well) were diluted appropriately and added to the plate, followed by the addition of the horse-radish peroxidase-conjugated monoclonal antibody (100 μl/well) diluted in assay buffer and incubated for 1 hour at 20° C. in the dark while shaking at 300 rpm. Finally, BM Chemiluminescence ELISA Substrate (Merck, CAT no. 11582950001) was added to each well and incubated for 3 minutes. The plates were then read using a fluorescent plate reader (Fluroskan FL, Thermo Fisher), with a read light emission at 1.000 ms and no filter. Standard curves were plotted using a four-parametric mathematical fit model.

### Technical Evaluation

### Proof-of-Concept Cleavage

Due to the complex structure of collagen molecules, their poor solubility, and tendency to assemble into fibrillar structures, synthetic peptides mimicking the type IV collagen alpha-3 and -4 chains were designed, with the target sequence located in the middle (Genscript). The neo-epitopes were then generated in vitro by incubating active neutrophil elastase purified from human cells by SDS-page (Abcam, cat. no. ab280938) with the synthetic peptides. Samples were incubated in a 50 mM Tris buffer with 500 mM NaCl (pH 7.5) for 4 hours at 37° C., in a reaction volume of 200 μl. Protein to protease ratio was 100:1. The proteolytic reaction was inhibited by adding the serine protease inhibitor AEBSF to a final concentration of 1 mM. Serving as experimental controls were samples containing only human neutrophil elastase (HNE), synthetic peptide without HNE, or only Tris buffer.

### Technical Validation

Technical validation of the assays was performed to evaluate robustness, accuracy, precision, interference and stability. The lower limit of blank (LLOB) was determined by the measurement of 60 assay buffer replicates. LLOB is defined as ±3×SD of buffer (blank) with no acceptance criteria. The lower limit of quantification (LLOQ) is defined as the lowest analyte concentration in serum where the CV % of the precision profile equals 25%. LLOQ was determined by measuring four low-level human serum samples in three replicates, in five separate runs. The upper limit of quantification (ULOQ) is defined as the highest standard point of the assay standard curve with acceptable accuracy and precision. ULOQ was determined by 10 independent runs of the standard curve. The standard curve robustness, and the half-maximal inhibition concentration (IC50) were also determined. The analytical measurement range is defined as the range between LLOQ and ULOQ. To assess linearity, four human serum samples were diluted from two-fold down to an eight-fold dilution. The percentage recovery from the undiluted sample was calculated, with an acceptance criterion of 100%±25. The reportable measurement range is based on the accepted dilution recovery and is defined as the range between LLOQ and ULOQ corrected for the maximum validated dilution. The inter- and intra-assay variations were determined by ten independent runs of eight human serum samples covering the quantifiable measurement range of the assays and two kit controls, all in double determinations. Acceptance criteria for inter-intra variability was a CV %<15.

To evaluate the accuracy of the assay and any potential matrix effects, peptide spiking recovery and matrix-matrix spiking recovery was determined. Serial diluted standard peptide in buffer was spiked into three sperate serum samples, and three sets of serum samples with high and low analyte concentrations were spiked into each other in different proportions. Recovery percentage was calculated based on expected and measured concentrations, with an RE % criterion of ±25.

Analyte stability in serum was assessed by exposing three human serum samples to four freeze and thaw cycles. The percentage recovery was calculated using the respective uncycled serum sample as a reference. To test whether common substances found in blood interfered with the assay analytes, an interference panel of biotin (0-100 ng/ml), hemoglobin (low=2.5 mg/ml, high=5 mg/ml) and lipid (low=1.5 mg/ml, high=5 mg/ml) was spiked into three individual human serum samples. The recovery percentage of the analyte for each interference sample was calculated with the respective control sample as reference, with an acceptance criterion of 100%±20.

### Clinical Evaluation

### Patient Samples

Patient serum samples from healthy subjects and from patients with CD or UC were obtained from ProteoGenix. Patient serum samples were also obtained from Odense University Hospital (OUH), Denmark. The OUH study subjects were enrolled in a prospective observational study (ClinicalTrials.gov ID: NCT02612103), including patients with CD (n=60), UC (n=101). Before sample collection, informed signed consent was collected from each study subject. The study was approved by the Regional Ethics Committee of Southern Denmark (journal number: S-20150107) and conducted according to the Declaration of Helsinki. Assessment of disease activity was based on the Harvey Bradshaw Index (HBI) for CD (0-4: remission; 5-7: mild activity; 8-16: moderate activity; >16: severe activity) and the partial Mayo score for UC ((<2: remission; 2-4: mild activity; 5-7: moderate activity; >7: severe activity). All patients were classified according to the Montreal classifications. The endoscopic score for disease activity was recorded for some of the IBD patients.

To assess endoscopic disease activity, The Simple Endoscopic Score was applied for CD (SES-CD) (0-2: remission; 3-6: mild disease activity; 7-15: moderate disease activity, >16: severe disease activity) and the Mayo endoscopic subscore was applied for UC (0: inactive disease; 1: mild activity, 2: moderate activity, 3: severe activity).

In addition, a further set of human serum samples (“Cohort 2”), including serum samples from patients with CD (n=72) and UC (n=28), was obtained from the Clinical Hospital Centre Zagreb (Department of Gastroenterology and Hepatology). The study was conducted according to the Declaration of Helsinki. Patients with co-morbidities and extra-intestinal manifestations were excluded from the study. Demographical data and disease activity were obtained from electronic medical records and questionnaires. Endoscopy was performed within three months of blood sampling. Endoscopic disease activity for UC was graded using the Mayo endoscopic score. The endoscopic scores were prospectively validated based on routine endoscopy and scoring by an experienced IBD endoscopist.

### Statistical Analysis

All data was considered non-parametric after visual assessment of normality using density plots. Differences in clinical data were compared using Mann-Whitney U-tests or Kruskal-Wallis with Dunn's correction for multiple comparisons. Receiver operating characteristics (ROC) statistics with the area under the curve (AUC) as an overall measure of fit were used to assess the discriminative capacity of the biomarkers. Sensitivity and specificity metrics were obtained by determining the optimal cut-off using Youden's J statistic. AUC values were presented with their corresponding 95% confidence intervals (CI). Data visualization was performed using Rstudio (version 4.1.2; Rstudio, Boston, MA, United States) and GraphPad Prism 9.2.0 (GraphPad software, San Diego, CA, United States). P-values <0.05 were considered statistically significant.

### Results

The C4A3-HNE and C4A4-HNE ELISA assays are highly specific

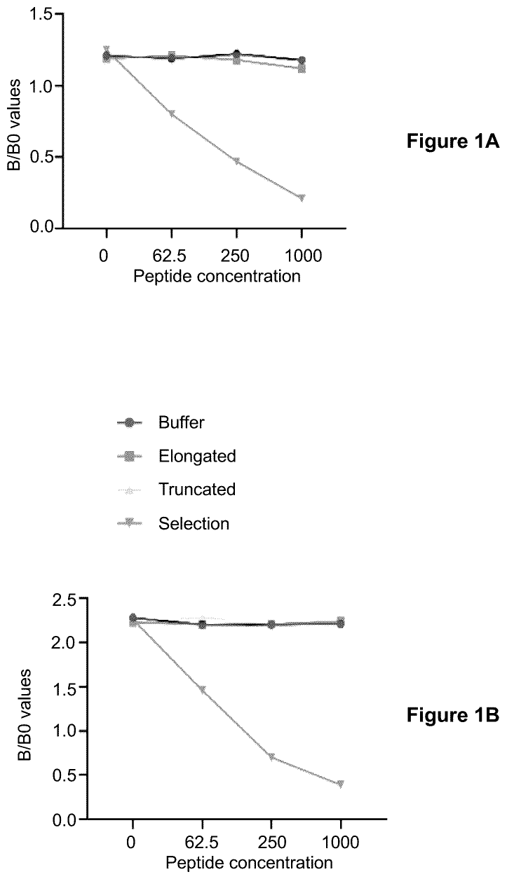

The specificity of the monoclonal antibodies raised against the C4A3-HNE (FIG. 1A) and C4A4-HNE (FIG. 1B) target sequences, was evaluated. For both assays the selection peptide representing the target sequence inhibited the signal in a dose-dependent manner. When a mismatch was introduced by using the elongated and truncated peptides, the signal inhibition was on par to the blank (no reactivity). This data shows that both monoclonal antibodies and the ELISAs using them are highly specific to the respective neo-epitopes that are being targeted.

The neo-epitopes represented by the C4A3-HNE and C4A4-HNE target sequences are released by HNE in vivo and can subsequently be detected by their respective assays

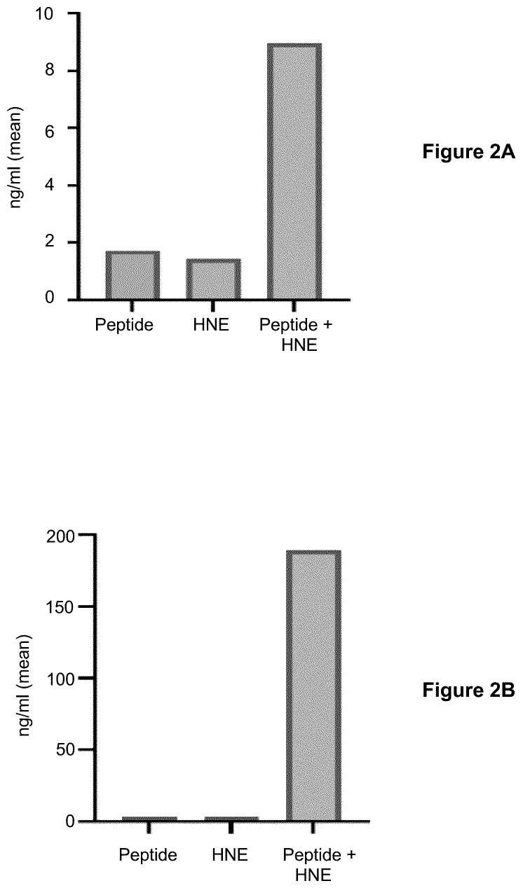

The ability of HNE to release the neo-epitopes was investigated in vivo by incubating synthetic peptides mimicking the type IV collagen alpha-3 and -4 chains, with active HNE for 4 hours. The results showed that HNE was able to generate both neo-epitopes (FIGS. 2A&B). Furthermore, the neo-epitopes were not detected in the undigested samples (only peptide).

### C4A3-HNE is a Technically Robust ELISA Assay

The technical performance of the C4A3-HNE assay in serum was further assessed through different technical validation steps, summarized in table 1. Intra-assay precision (CV %) was determined to be ≤13% and inter-assay precision (CV %) was determined to be ≤98. Accuracy (% RE) was determined to be in the range of −4%-22%. The linearity study showed acceptable dilution recovery up to 25% dilution. Based on these data, maximum validated dilution of human serum samples is a dilution of 1+3. The lower limit of blank (LLOB) was determined to be 0.6 ng/ml. Furthermore, the C4A3-HNE analyte showed to be stable for up to five freeze/thaw cycles in human serum. No interference was detected from low or high contents of biotin, lipemia or hemoglobin in serum with recoveries ranging from 82-108%. Taken together, these results indicate that the C4A3-HNE assay is a technically robust assay and can used for testing of human serum samples.

TABLE 1
Technical validation parameters of C4A3-HNE

| PARAMETER | C4A3-HNE | |
|---|---|---|
| Measurement range in serum (LLOQ-ULOQ) | 15.6-1000.0 | (ng/ml) |
| Lower limit of blank (LLOB) | 0.56 | ng/ml |
| Mean IC50 | 73.12 | ng/ml |

| | |
|---|---|
| Mean Slope | 1.01 |
| Spiking recovery of serum in serum | −4%-22% |
| Intra-assay variation (CV %) | ≤13% |
| Inter-assay variation (CV %) | ≤9% |
| Dilution recovery (1 + 3) | 88-122% |
| Freeze/thaw recovery (5 cycles) | 77-114% |
| Biotin recovery, low/high^{a} | 98%/82% |
| Lipemia recovery, low/high^{b} | 108%/103% |
| Hemoglobin recovery, low/high^{c} | 104%/88.9% |

^{a}2.5/5 mg/ml,
^{b}1.5/5 mg/ml,
^{c}5/80 ng/ml

The technical performance of the C4A4-HNE assay in serum was assessed through different technical validation steps, summarized in table 2. Intra-assay precision (CV8) was determined to be ≤7% and inter-assay precision (CV %) was determined to be ≤12%. Accuracy (% RE) was determined to be in the range of −3%-15%. The linearity study showed acceptable dilution recovery up to 25% dilution. Based on these data, maximum validated dilution of human serum samples is a dilution of 1+3. The lower limit of blank (LLOB) was determined to be 1.36 ng/ml. Furthermore, the C4A4-HNE analyte showed to be stable for up to five freeze/thaw cycles in human serum. No interference was detected from low or high contents of biotin, lipemia or hemoglobin in serum with recoveries ranging from 82-1178. These results indicate that C4A4-HNE is a technically robust assay and can used for testing of human serum samples.

TABLE 2
Technical validation parameters of C4A4-HNE

| PARAMETER | C4A4-HNE | |
|---|---|---|
| Measurement range in serum (LLOQ-ULOQ) | 5.62-444.44 | (ng/ml) |
| Lower limit of blank (LLOB) | 1.36 | ng/ml |
| Mean IC50 | 33.55 | ng/ml |

| | |
|---|---|
| Mean Slope | 0.95 |
| Spiking recovery of serum in serum | −3-15% |
| Intra-assay variation (CV %) | ≤7% |
| Inter-assay variation (CV %) | ≤12% |
| Dilution recovery (1 + 3) | 80-109% |
| Freeze/thaw recovery (5 cycles) | 86-105% |
| Biotin recovery, low/high^{a} | 112%/82% |
| Lipemia recovery, low/high^{b} | 115%/117% |
| Hemoglobin recovery, low/high^{c} | 112%/95% |

^{a}5/100 ng/ml,
^{c}1.5/5 mg/ml,
^{d}2.5/5 mg/ml

### C4A3-HNE is Elevated in IBD Compared to Healthy

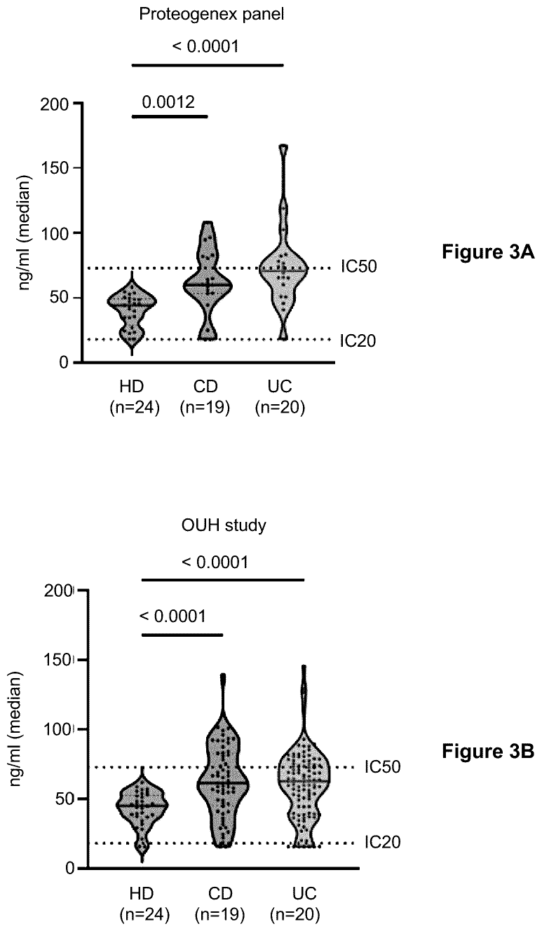

The biological relevance of C4A3-HNE was assessed by measuring the levels of C4A3-HNE, using the C4A3-HNE ELISA, in serum samples from IBD patients and healthy donors (obtained from ProteoGenix). As shown in FIG. 3A, C4A3-HNE was elevated in both CD and UC compared to healthy (p=0.001 and <0.0001) (AUC [95% CI]: 0.820 [0.669-0.971]; 0.875 [0.775-1.000]). These results were validated in the serum samples from the OUH cohort of IBD patients (both p<0.0001), as shown in FIG. 3B.

C4A3-HNE is elevated in IBD patients with mild endoscopic activity

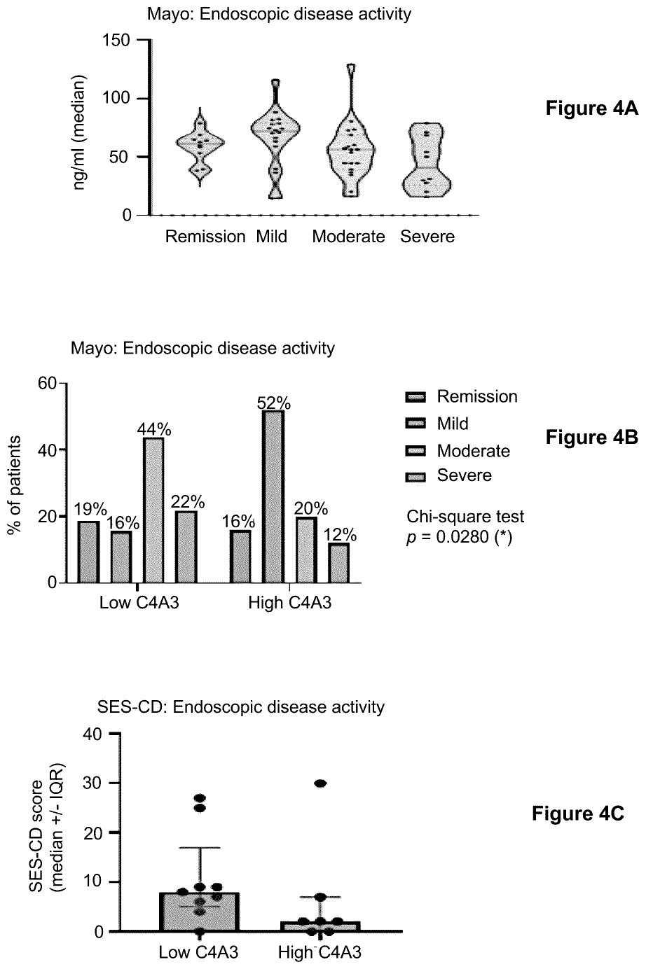

When looking ulcerative colitis patients stratified by their endoscopic disease severity score, a pattern can be seen in the levels of C4A3-HNE. The biomarker levels are highest in patients with mild endoscopic disease (71.77 ng/ml), and the levels decrease dose-dependently for patients with moderate (56.07 ng/ml) and severe (40.52 ng/ml) endoscopic disease (FIG. 4A). Patients were then divided into two groups based on whether they were above or below median C4A3-HNE levels (62.75 ng/ml). Patients with low levels of the biomarker mostly have moderate (44%) and severe (22%) disease, as opposed to patients with high levels mainly having mild (52%) disease (FIG. 4B). Taken together, C4A3-HNE decreases dose-dependently from patients with mild to moderate/severe endoscopic disease. Not enough patients with CD had registered endoscopic disease scores, so grouping them according to disease severity was not feasible. Grouping the patients based on their median C4A3-HNE levels (61.24 ng/ml) revealed that those with low levels of C4A3-HNE had higher median endoscopic disease activity scores, compared to those who had high levels of the marker (FIG. 4C). These results show a similar pattern as seen in patients with ulcerative colitis, indicating that high level of the C4A3-HNE biomarker reflect early mucosal damage in patients with IBD.

Inflammatory markers are lower in IBD patients with high levels of C4A3-HNE

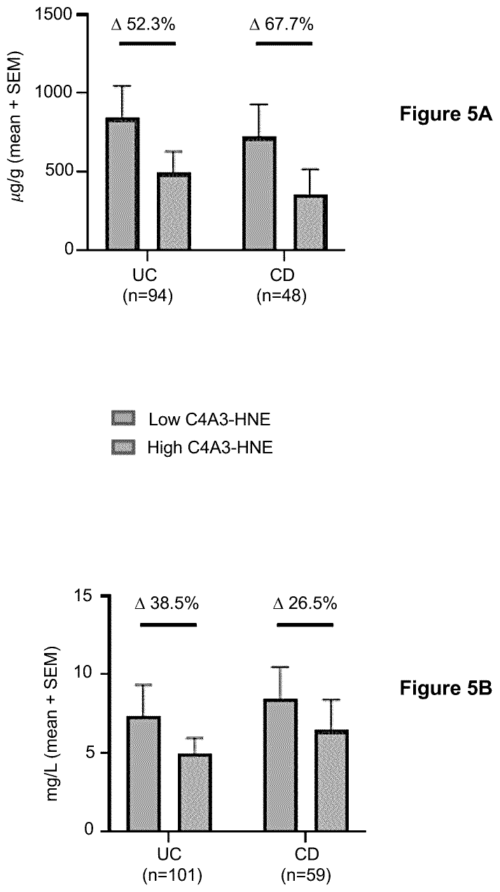

Patients with CD and UC were again grouped according to low/high C4A3-HNE values, based on the median for each group. Mean fecal calprotectin and C-reactive protein (CRP) levels were assessed between the groups. Results showed that IBD patients with low levels of C4A3-HNE had numerically lower mean levels of both fecal calprotectin and CRP compared to patients with high biomarker levels (FeCal (μg/g): UC-840.0 vs. 491.6/CD-721.8 vs. 356.7) (CRP (ml/L): UC-7.37 vs. 4.99/CD-8.34 vs 6.45) (FIG. 5). Taken together, this data points towards high levels of C4A3-HNE being indicative of early disease.

C4A3-HNE does not reflect the same pathological process as an MMP-generated biomarker of the same alpha-chain

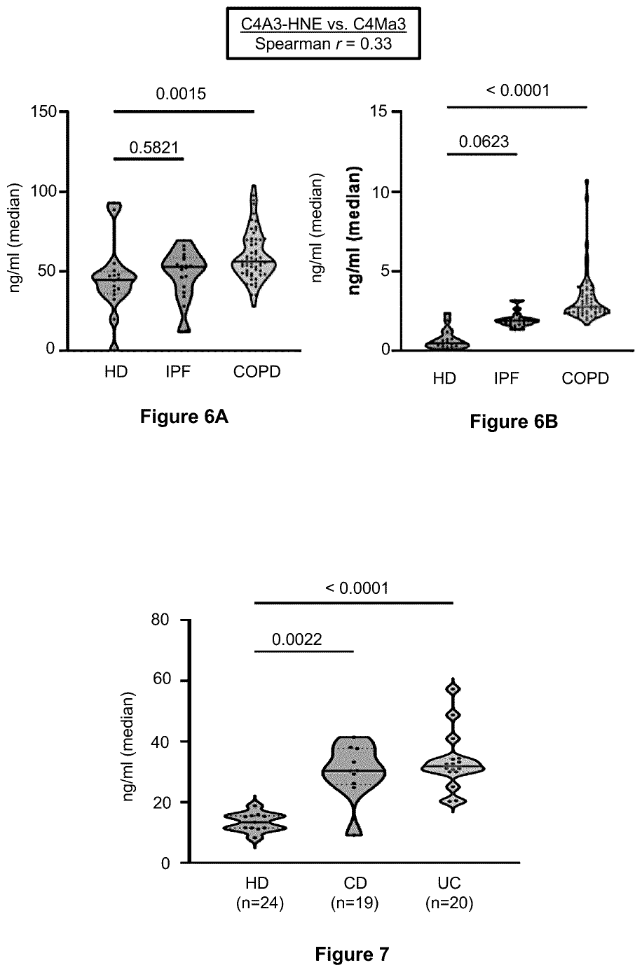

To emphasize the uniqueness of the C4A3-HNE marker, we compared the marker to another type IV alpha-3 collagen neo-epitope biomarker (C4Ma3). The biomarker in question is generated by MMPs and is therefore reflective of lymphocyte activity, as opposed to neutrophil activity. The biomarker was developed to evaluate fibrosis, mainly in lung disease since the alpha-3 chain was long thought to be kidney- and lung specific ^{[14]}. To compare the two biomarkers, a cohort of patients with idiopathic pulmonary fibrosis (IPF) and chronic obstructive pulmonary disease (COPD) were measured. Results showed that both biomarkers were elevated compared to healthy, but upon Spearman correlation analysis we saw that they only had a weak correlation of 0.33 (FIG. 6). In other words, C4A3-HNE does not reflect the same pathological process as C4Ma3 as they differ ~70%.

C4A4-HNE is also elevated in IBD compared to healthy

The biological relevance of C4A4-HNE was also assessed by measuring the levels of C4A4-HNE, using the C4A4-HNE ELISA, in serum samples from IBD patients and healthy donors (obtained from ProteoGenix). As shown in FIG. 7, C4A4-HNE is elevated in IBD patients compared to healthy (p<0.01).

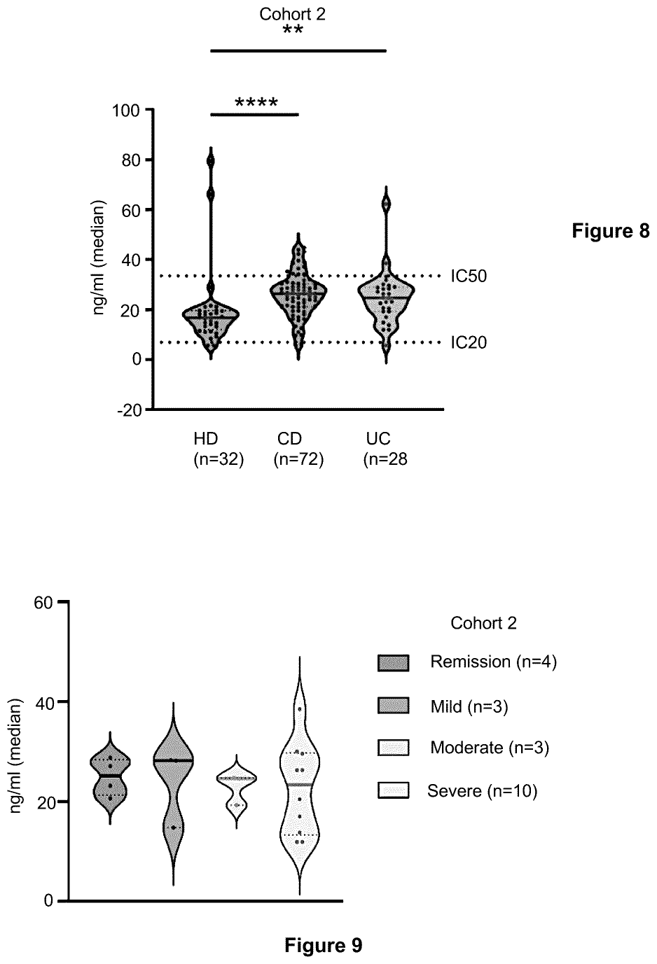

The biological relevance of C4A4-HNE was further validated in a further cohort of IBD patients (Cohort 2). As shown in FIG. 8, C4A4-HNE is elevated in both CD and UC compared with healthy donors (HD vs. CD vs. UC [IQR]: 16.68 ng/ml [11.93, 19.55] vs. 26.33 ng/ml [21.05, 30.30] vs. 24.66 ng/ml [19.24, 28.93], p<0.0001 and <0.01) (AUC [95% CI]: 0.91 [0.70-0.99]; 1.00 [0.87-1.00]) (FIG. 8).

C4A4-HNE dose-dependently decreases from patients with mild endoscopic disease to patients with severe endoscopic disease

Patients with UC in Cohort 2 were grouped according to their endoscopic disease severity score, where a similar pattern was noticed for levels of C4A4-HNE as with levels of C4A3-HNE. As shown in FIG. 9, patients with mild endoscopic disease had the highest levels of C4A4-HNE (ng/ml [IQR]: 28.2 [21.5, 28.2]) with a dose-dependent decrease across severity groups (ng/ml [IQR]: 24.6 [21.9, 24.7]; 23.3 [14.6, 28.8]).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1] C. McDowell, U. Farooq, M. Haseeb, Inflammatory Bowel Disease, 2022. http://www.ncbi.nlm.nih.gov/pubmed/30137275.
[2] S. H. Lee, J. eun Kwon, M. la Cho, Immunological pathogenesis of inflammatory bowel disease, Intest Res. 16 (2018) 26-42. https://doi.org/10.5217/ir.2018.16.1.26.
[3] B. E. Sands, B. G. Feagan, P. Rutgeerts, J. F. Colombel, W. J. Sandborn, R. Sy, G. D'Haens, S. Ben-Horin, J. Xu, M. Rosario, I. Fox, A. Parikh, C. Milch, S. Hanauer, Effects of vedolizumab induction therapy for patients with Crohn's disease in whom tumor necrosis factor antagonist treatment failed, Gastroenterology. 147 (2014) 618-627.e3. https://doi.org/10.1053/j.gastro.2014.05.008.
[4] B. Drury, G. Hardisty, R. D. Gray, G. tzer Ho, Neutrophil Extracellular Traps in Inflammatory Bowel Disease: Pathogenic Mechanisms and Clinical Translation, Cmgh. 12 (2021) 321-333. https://doi.org/10.1016/j.jcmgh.2021.03.002.
[5] W. Fischbach, W. Becker, J. Mossner, H. Ohlemüller, W. Koch, W. Borner, Leucocyte elastase in chronic inflammatory bowel diseases: A marker of inflammatory activity?, Digestion. 37 (1987) 88-95. https://doi.org/10.1159/000199473.
[6] S. Kirov, A. Sasson, C. Zhang, S. Chasalow, A. Dongre, H. Steen, A. Stensballe, V. Andersen, S. Birkelund, T. B. Bennike, Degradation of the extracellular matrix is part of the pathology of ulcerative colitis, Mol Omics. 15 (2019) 67-76. https://doi.org/10.1039/c8mo00239h.
[7] S. K. Jorch, P. Kubes, An emerging role for neutrophil extracellular traps in noninfectious disease, 23 (2017) 279-287. https://doi.org/10.1038/nm.4294.
[8] J. H. Mortensen, M. Lindholm, L. L. Langholm, J. Kjeldsen, A. C. Bay-Jensen, M. A. Karsdal, T. Manon-Jensen, The intestinal tissue homeostasis—the role of extracellular matrix remodeling in inflammatory bowel disease, Expert Rev Gastroenterol Hepatol. 13 (2019) 977-993. https://doi.org/10.1080/17474124.2019.1673729.
[9] H. P. Bachinger, K. Mizuno, J. A. Vranka, S. P. Boudko, Collagen formation and structure, Comprehensive Natural Products II: Chemistry and Biology. 5 (2010) 469-530. https://doi.org/10.1016/b978-008045382-8.00698-5.
[10] Y. Oka, I. Naito, K. Manabe, Y. Sado, H. Matsushima, Y. Ninomiya, M. Mizuno, T. Tsuji, Distribution of collagen type IV α1-6 chains in human normal colorectum and colorectal cancer demonstrated by immunofluorescence staining using chain-specific epitope-defined monoclonal antibodies, Journal of Gastroenterology and Hepatology (Australia). 17 (2002) 980-986. https://doi.org/10.1046/j.1440-1746.2002.02789.x.
[11] H. Sato, I. Naito, R. Momota, Y. Naomoto, T. Yamatsuji, Y. Sado, Y. Ninomiya, A. Ohtsuka, The differential distribution of type IV collagen a chains in the subepithelial basement membrane of the human alimentary canal, Arch Histol Cytol. 70 (2007) 313-323. https://doi.org/10.1679/aohc.70.313.
[12] N. Maillet, Rapid Peptides Generator: fast and efficient in silico protein digestion., NAR Genom Bioinform. 2 (2020) lqz004. https://doi.org/10.1093/nargab/lqz004.
[13] M. L. Gefter, D. H. Margulies, M. D. Scharff, A simple method for polyethylene glycol-promoted hybridization of mouse myeloma cells., Somatic Cell Genet. 3 (1977) 231-6. https://doi.org/10.1007/BF01551818.
[14] J. M. Sand, L. Larsen, C. Hogaboam, F. Martinez, M. L. Han, M. R. Larsen, A. Nawrocki, Q. Zheng, M. A. Karsdal, D. J. Leeming, MMP mediated degradation of type IV collagen alpha 1 and alpha 3 chains reflects basement membrane remodeling in experimental and clinical fibrosis-Validation of two novel biomarker assays, PLOS One. 8 (2013) 1-12. https://doi.org/10.1371/journal.pone.0084934.

1: A method of immunoassay, said method comprising;
i) contacting a patient sample with a monoclonal antibody that specifically binds to the N-terminus amino acid sequence EGTRPGPPGP (SEQ ID NO: 1) or that specifically binds to the N-terminus amino acid sequence TYPGRHGPPG (SEQ ID NO: 2); and
ii) detecting and determining the amount of binding between said monoclonal antibody and peptides in the sample.

2: The method as claimed in claim 1, wherein the method is a method of immunoassay for detecting and/or monitoring inflammatory bowel disease or a particular level of severity thereof in a patient, the method further comprising;
iii) correlating said amount of binding with values associated with normal healthy subjects and/or values associated with known disease severity and/or values obtained from said patient at a previous time point and/or with a predetermined cut-off value.

3: The method as claimed in claim 2, wherein the inflammatory bowel disease is Crohn's disease or ulcerative colitis.

4: The method as claimed in claim 1, wherein the patient sample is selected from blood, plasma or serum.

5: The method as claimed in claim 1, wherein the monoclonal antibody specifically binds to the N-terminus amino acid sequence EGTRPGPPGP (SEQ ID NO: 1) and does not specifically bind to a peptide having the N-terminus amino acid sequence SEGTRPGPPGP (SEQ ID NO: 3).

6: The method as claimed in claim 1, wherein the monoclonal antibody specifically binds to the N-terminus amino acid sequence EGTRPGPPGP (SEQ ID NO: 1) and does not specifically bind to a peptide having the N-terminus amino acid sequence GTRPGPPGP (SEQ ID NO: 4).

7: The method as claimed in claim 1, wherein the monoclonal antibody is raised against a synthetic peptide having the N-terminus amino acid sequence EGTRPGPPGP (SEQ ID NO: 1).

8: The A method as claimed in claim 1, wherein the monoclonal antibody specifically binds to the N-terminus amino acid sequence TYPGRHGPPG (SEQ ID NO: 2) and does not specifically bind to a peptide having the N-terminus amino acid sequence VTYPGRHGPPG (SEQ ID NO: 5).

9: The method as claimed in claim 1, wherein the monoclonal antibody specifically binds to the N-terminus amino acid sequence TYPGRHGPPG (SEQ ID NO: 2) and does not specifically bind to a peptide having the N-terminus amino acid sequence YPGRHGPPG (SEQ ID NO: 6).

10: The method as claimed in claim 1, wherein the monoclonal antibody is raised against a synthetic peptide having the N-terminus amino acid sequence TYPGRHGPPG (SEQ ID NO: 2).

11: The method as claimed in claim 1, wherein the immunoassay is a competition assay or a sandwich assay.

12: The A method as claimed in claim 1, wherein the immunoassay is a radio-immunoassay or an enzyme-linked immunosorbent assay.

13: A monoclonal antibody that specifically recognises the N-terminus amino acid sequence EGTRPGPPGP (SEQ ID NO: 1) or that specifically recognises and binds to the N-terminus amino acid sequence TYPGRHGPPG (SEQ ID NO: 2).

14: The monoclonal antibody as claimed in claim 13, wherein the monoclonal antibody specifically binds to the N-terminus amino acid sequence EGTRPGPPGP (SEQ ID NO: 1) and does not specifically bind to a peptide having the N-terminus amino acid sequence SEGTRPGPPGP (SEQ ID NO: 3).

15: The monoclonal antibody as claimed in claim 13, wherein the monoclonal antibody specifically binds to the N-terminus amino acid sequence EGTRPGPPGP (SEQ ID NO: 1) and does not specifically bind to a peptide having the N-terminus amino acid sequence GTRPGPPGP (SEQ ID NO: 4).

16: The monoclonal antibody as claimed in claim 13, wherein the monoclonal antibody is raised against a synthetic peptide having the N-terminus amino acid sequence EGTRPGPPGP (SEQ ID NO: 1).

17: The monoclonal antibody as claimed in claim 13, wherein the monoclonal antibody specifically binds to the N-terminus amino acid sequence TYPGRHGPPG (SEQ ID NO: 2) and does not specifically bind to a peptide having the N-terminus amino acid sequence VTYPGRHGPPG (SEQ ID NO: 5).

18: The monoclonal antibody as claimed in claim 13, wherein the monoclonal antibody specifically binds to the N-terminus amino acid sequence TYPGRHGPPG (SEQ ID NO: 2) and does not specifically bind to a peptide having the N-terminus amino acid sequence YPGRHGPPG (SEQ ID NO: 6).

19: The monoclonal antibody as claimed in claim 13, wherein the monoclonal antibody is raised against a synthetic peptide having the N-terminus amino acid sequence TYPGRHGPPG (SEQ ID NO: 2).

20: An immunoassay kit comprising a monoclonal antibody as claimed in claim 13, and at least one of:
a streptavidin coated well plate;
a biotinylated peptide EGTRPGPPGP-L-Biotin (SEQ ID NO: 7), wherein L is an optional linker or a biotinylated peptide TYPGRHGPPG-L-Biotin (SEQ ID NO: 8) wherein L is an optional linker;
a secondary antibody for use in a sandwich immunoassay;
a calibrator protein comprising the N-terminus amino acid sequence EGTRPGPPGP (SEQ ID NO: 1) or a calibrator protein comprising the N-terminus amino acid sequence TYPGRHGPPG (SEQ ID NO: 2);
an antibody biotinylation kit;
an antibody HRP labelling kit; or
an antibody radiolabelling kit.
